(19) United States
(12) Patent Application Publication
(10) Pub. No.: US 2016/0066563 A1
(43) Pub. Date: Mar. 10, 2016

(54) COMPOSITIONS AND METHODS FOR COLLECTING, WASHING, CRYOPRESERVING, RECOVERING AND RETURN OF LIPOASPIRATES TO PHYSICIAN FOR AUTOLOGOUS ADIPOSE TRANSFER PROCEDURES

(71) Applicant: David MOSCATELLO, (US)

(72) Inventor: David MOSCATELLO, Philadelphia, PA (US)

(21) Appl. No.: 14/406,203

(22) PCT Filed: Jun. 6, 2013
(86) PCT No.: PCT/US13/44621

§ 371 Date: Dec. 5, 2014

Related U.S. Application Data
(60) Provisional application No. 61/656,837, filed on Jun. 7, 2012.

(51) Int. Cl. A01N 1/02; C12N 5/077

(52) U.S. Cl. CPC A01N 1/0221; C12N 5/0653

(57) ABSTRACT

The invention details a cryoprotectant and the methods to prepare biological tissue and related business methods and systems. The invention is directed to methods for collecting, washing, cyropreserving, recovering, and return of lipoaspirates to physicians for autologous adipose tissue transfer procedures in patients. In an embodiment, the invention is directed to a cryoprotectant solution for cryopreserving biological tissue essentially of a polyol and a crysalloid.

### FIELD OF THE INVENTION

The invention is directed to method for collecting, washing, cryopreserving, recovering, and return of lipoaspirates to physicians for autologous adipose tissue transfer procedures in patients.

### BACKGROUND OF THE INVENTION

There still exists today the need for a method for cryopreserving aspirated adipose tissue in a form suitable for reinjection into a patient upon thawing. While physicians have been performing autologous adipose tissue transfer procedures for decades, the procedures are not standardized, and results are often sub-optimal. This results in the need for repeat procedures, and physicians sometimes freeze excess lipoaspirate for subsequent use. However, without cryoprotectants and proper storage processes and temperatures, the viability of such tissue is lost (Lidagoster et al., 2000; Ullman et al., 2004; Moscatello et al., 2005; Wolter et al., 2005). While a number of cryopreservation solutions and methods have been tested for freezing adipose tissue, e.g. dimethylsulfoxide (DMSO) and fetal bovine serum (FBS), none have used reagents suitable for direct clinical use in humans (Pu et al., 2007; Cui et al., 2007; Pu el al., 2010).

### BRIEF SUMMARY OF THE INVENTION

In a first embodiment, the invention is directed to a cryoprotectant solution for cryopreserving biological tissue essentially of a polyol and a crystalloid.

In another embodiment, the invention is directed to a system for cryopreserving biological tissue including a cryoprotectant solution which does not cause leaching from a plastic based containers and a plastic based container.

In another embodiment, the invention is directed to a method for collecting, washing, cryopreserving, recovering, and return of lipoaspirates to physicians for autologous adipose tissue transfer procedures. All reagents are suitable for clinical use according to United States Pharmacopea (USP) and the collection containers and accessories used all have U.S. Food and Drug Administration (FDA) approval for clinical uses. The method is designed to obtain high percent viability of adipocytes after cryopreservation and thawing of adipose tissue including the steps of obtaining an adipose tissue specimen and washing the adipose tissue with a wash solution comprising Lactated Ringer's solution.

In a further embodiment the invention is directed to a method cyropreserve adipose tissue including the steps of obtaining an adipose tissue specimen and washing the adipose tissue with a wash solution of a crystalloid solution. The wash is removed wherein a cryoprotectant solution including a polyol and a crystalloid equal to the volume of adipose tissue to be preserved is added. The infranatant solution is separated and removed. The infranatant solution is tested for microbial contamination. The cryoprotected adipose tissue is placed into multiple containers; and the cryoprotected adipose tissue is cryopreserved at a defined rate. The cryopreserved adipose tissue is stored at a temperature below −150 degrees Celsius.

In yet another embodiment the invention is directed to a business method for collection, cryogenic storage and distribution of an autologous biological sample material.

### BRIEF DESCRIPTION OF THE DRAWINGS

FIG. 1 is a flow chart of the basic cryopreservation method of the present invention.

### DETAILED DESCRIPTION OF THE INVENTION

Certain terminology is used herein for convenience only and is not to be taken as a limitation on the present invention. The terminology includes the words specifically mentioned, derivatives thereof and words of similar import. The embodiments discussed herein are not intended to be exhaustive or to limit the invention to the precise form disclosed. These embodiments are chosen and described to best explain the principle of the invention and its application and practical use and to enable others skilled in the art to best utilize the invention.

In a first embodiment, the invention is directed to a cryoprotectant solution for cryopreserving biological tissue essentially of a polyol and a crystalloid. In a preferred embodiment the polyol is glycerol and the crystalloid is Lactated Ringers solution. Without limiting the concept of the present invention, the polyol and the crystalloid are selected in this embodiment for their interaction with a biological tissue such as adipose tissue.

The glycerol and Lactated Ringers are in a ratio of approximately 1 to 10 respectively. This solution establishes equilibration within 20 minutes after combination with adipose tissue. Equilibration means the time to obtain a steady state concentration inside and outside the cells to be equal. The combination of glycerol and Lactated Ringers in a ratio of approximately 1 to 10 respectively in the present embodiment provides a highly effective cryoprotectant that can be directly injected into a patient for use in cosmetic or surgical procedures. Those skilled in the art will recognize that combinations of glycerol and Lactated Ringer's in ratios between 1 to 20 and 1 to 10 are not to be considered significantly different in the cryopreservation of adipose tissue but the lowest acceptable ratio is 1 to 40 (2.5%). Further, the cryoprotectant solution of the present invention does not require any type of digestion of the adipose tissue of the present embodiment Thus, use of the cryoprotectant of the present invention provides a safe, efficient and cost effective alternative to cryoprotectants currently available.

In another embodiment, the invention is directed to a system for cryopreserving biological tissue including a cryoprotectant solution which does not cause leaching from a plastic based container. As it is known in the art that DMSO is a common cryoprotectant, it is also recognized that is has two significant liabilities. First, DMSO causes “leaching” from most “plastic” type bags and therefore, requires bags or containers that are of a special formulation and are expensive. Second, and more importantly, DMSO is toxic to the body and cannot be injected into the body in significant amounts (as discussed herein).

The cryoprotectant solution of the system of the present embodiment is focused on (i) a polyol and (ii) a crystalloid. As discussed in the previous embodiment, in the present embodiment the polyol is glycerol and the crystalloid is Lactated Ringers solution. Thus, in contrast to any solutions which contain DMSO, the system of the present embodiment provides for the direct injection of the cryopreserved adipose tissue into the body and also is cost effective. This is due to the recognition that all components of the cryoprotectant of the present invention are non-toxic and approved by the Food and Drug Administration (FDA) for use in patients; more specifically. Lactated Ringer's Injection, is a U.S. Pharmacopeia (USP) sterile, nonpyrogenic solution for fluid and electrolyte replenishment in single dose containers for intravenous administration and Glycerine is classified by the U.S. Food and Drug Administration (FDA) as “generally recognized as safe” (GRAS). The flexible container is made with non-latex plastic materials specially designed for a wide range of parenteral drugs including those requiring delivery in containers made of polyolefins or polypropylene. The solution contact materials do not contain PVC, DEHP, or other plasticizers. The suitability of the container materials has been established through biological evaluations, which have shown the container passes Class VI USP testing for plastic containers. These tests confirm the biological safety of the container system. Having the ability to ship the “thawed” cryopreserved adipose tissue to a physician who can inject it directly into a patient reduces cost, increases efficiency of the procedure and reduces (and potentially) eliminates contamination.

The plastic based container has at least one tube port with a Luer fitting and at least one spike port. This is required to effectuate the direct transfer of the cryopreserved adipose tissue (now thawed) as discussed herein. Specifically, upon receipt of the cryopreserved adipose tissue (now thawed) the physician simply extracts this tissue via a syringe and directly injects this tissue into the desired area of a patient.

In another embodiment the invention is directed to a method to obtain a high percent viability of adipocytes after cryopreservation and thawing of adipose tissue. The method includes the steps of obtaining an adipose tissue specimen and washing the adipose tissue with a wash solution including Lactated Ringer's solution. A solution of glycerol in Lactated Ringer's is added to the washed adipose tissue to obtain glycerol cryoprotected adipose tissue in the original container and at least one second container. The glycerol cryoprotected adipose tissue is cryopreserved by cooling at a controlled rate, and stored at temperatures below −80 degrees Celsius (C). In a preferred method, the storage temperature will be maintained below −150 degrees Celsius in the vapor phase of a tank containing liquid nitrogen.

The cryopreserved glycerol cryoprotected adipose tissue is thawed in a liquid bath at a temperature of approximately 35 to 40 degrees Celsius; (37 degrees Celsius in a preferred method) to form a recovered glycerol protected adipose tissue. Lactated Ringers solution is added in an amount approximately equal to the volume of glycerol protected adipose tissue in the container to form a suspension solution. The suspension is separated to form (i) infranatants and (ii) adipose tissue; removing the infranatant solution from the container. The recovered adipose tissue is returned to the physician for use in a scheduled autologous adipose tissue transfer procedure.

As discussed, core to the present invention is the ability to directly inject the adipose tissue into a patient upon thawing in a safe and non-toxic cryoprotectant. The quality control steps of the method of the present embodiment are initiated by obtaining a quality-control aliquot of the same cryopreserved adipose tissue recovered, wherein Lactated Ringers in an amount substantially larger than the volume of adipose tissue is added in the quality control aliquot. The re-suspended, recovered adipose tissue is centrifuged to form (i) infranatant wash and (ii) washed, recovered adipose tissue. The aliquot of the washed, recovered adipose tissue is transferred to a tube containing collagenase solution. The adipose tissue-collagenase suspension is incubated at approximately 37 degrees C. to partially dissociate the adipose tissue into adipocytes and stromal-vascular fraction cells. The collagenase is neutralized by adding a growth medium to the adipose tissue-collagenase suspension, and the adipose tissue is recovered by centrifuging the digested, recovered adipose tissue to separate the floating adipocytes from free stromal-vascular fraction cells. A sample of the dissociated adipocytes is transferred from the recovered adipose tissue to a tube containing a vital stain to determine the percentage of viable adipocytes in the sample using an instrument capable of distinguishing live adipocytes from dead adipocytes based on the vital stain used. The results of the viability analysis are distributed to the collecting physician, who most commonly performs the procedure to inject the thawed adipose tissue into the patient. Using this method the percentage of viable adipose tissue cells is typically greater than 70.0 percent.

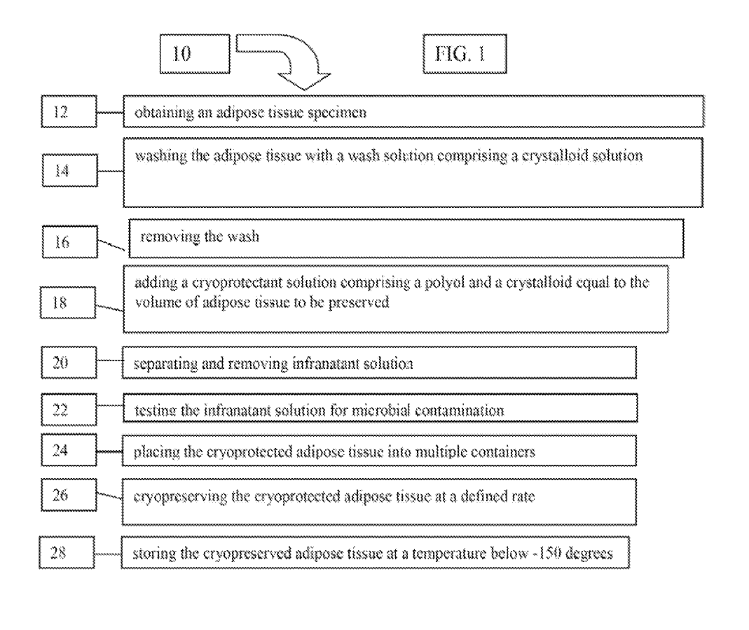

Referring to FIG. 1, in a further embodiment the invention is directed to a method to cyropreserve adipose tissue 10 including the steps of obtaining an adipose tissue specimen 12 and washing the adipose tissue with a wash solution of a crystalloid solution 14. The wash is removed 16 wherein a cryoprotectant solution including a polyol and a crystalloid equal to the volume of adipose tissue to be preserved is added 18. The infranatant solution is separated and removed 20. The infranatant solution is tested for microbial contamination 22.

The cryoprotected adipose tissue is placed into multiple containers 24 and the cryoprotected adipose tissue is cryopreserved at a defined rate. 26 The cryopreserved adipose tissue is stored at a temperature below −150 degrees Celsius. 28

The defined rate of cyropreserving is −1 degree Celsius/minute to at least −20 degrees Celsius, and cooling is continued at −1 to −2 degrees Celsius per minute to −80 degrees Celsius. The cooling rate subsequent to the phase transition (from liquid to solid) is less critical than the initial cooling rate. As appreciated in the present invention, the polyol is glycerol. And the crystalloid is Lactated Ringers solution.

In yet another embodiment the invention is directed to a business method for collection, cryogenic storage and distribution of an autologous biological sample material. The method is initiated by collecting a premium for defined services for collection, cryogenic storage and distribution of a biological sample material and thereafter coordinating the collection of a biological sample of a customer by (i) paying a predetermined fee in support of physician services performed for collection of the biological sample and (ii) supplying a collection system including a plurality of components for collection and transportation of the biological sample. This initial part of the business method is important not only to obtain the sample but to initiate the business relationship of the customer and business entity. The customer, physician and business entity will gain an understanding of the “big picture” and long-term relationship of this collaboration so as to appreciate the benefits, rights, obligations and costs (as explained herein).

The pre-determined fee for a physician to obtain the biological sample will vary depending upon the total volume of adipose tissue to be processed and cryopreserved, but will mostly likely be limited to costs relating to the collection system, transportation to the processing facility, and cryopreservation. However, the cost will be a one-time set fee which will be agreed upon by the client before initiating the procedure to obtain the sample.

The collection system is a defined set of components which are designed for coordination of the business method. The collection system includes an identification material for the obtained biological sample. This is most commonly a defined group of standard forms which may include coded labels for use with an encoded program (as discussed herein). Client sample bags include the same coded labels for use with the encoded program. These labels will comply with state and federal regulations, e.g. 21 CFR 11. The collection system further includes a transportation box which may be commercially manufactured and coordinated with a transportation carrier, e.g. FedEx. Transportation labeling will also include the same coded labels for use with the same encoded program; in addition to information regarding shipment location. Upon coordination, the method continues by obtaining the biological sample from the client and transporting the biological sample in the collection system to a processing facility.

At the process facility, the collection system components are introduced to a processing module of a database via a log-in port; having the encoded program. The database will be custom-designed to process and store eProtected health information using a proprietary program or a commercially available program such as Microsoft's Access program. The database will include but is not limited to, the information obtained from the collection system to coordinate the “client sample with the client”; such as the information included in the patient-specific bar-coded client sample bags. This information will also be included in a standardized form. The database will be organized in modules similar to the organization in the standardized form, will be searchable, and will be programmed to produce all the various forms associated with this process. The database includes the encoded program to organize and store information regarding the biological sample and recording information.

The biological sample is processed by washing the adipose tissue with a wash solution comprising Lactated Ringer's solution. A solution of glycerol in Lactated Ringer's is added to the washed adipose tissue to obtain glycerol cryoprotected adipose tissue in the original container and at least one second container. The glycerol cryoprotected adipose tissue is cryopreserved.

Upon request, the cryopreserved glycerol cryoprotected adipose tissue is thawed in a liquid bath at a temperature of approximately 37 degrees Celsius to form a recovered glycerol protected adipose tissue. Lactated Ringers solution is added in an amount approximately equal to the volume of glycerol protected adipose tissue in the container to form a suspension solution. The suspension is separated to form (i) infranatant and (ii) adipose tissue; the infranatant solution is removed from the container. The recovered adipose tissue is returned to the physician for use in a scheduled autologous adipose tissue transfer procedure.

A quality-control aliquot of the same cryopreserved adipose tissue is recovered and Lactated Ringers in an amount substantially larger than the volume of adipose tissue is added in the quality control aliquot. The re-suspended, recovered adipose tissue is centrifuged to form (i) infranatant wash and (ii) washed, recovered adipose tissue. The aliquot of the washed, recovered adipose tissue is transferred to a tube containing collagenase solution. The adipose tissue-collagenase suspension is incubated at approximately 37 degrees C. to partially dissociate the adipose tissue into adipocytes and stromal-vascular fraction cells. The collagenase is neutralized by adding a growth medium to the adipose tissue-collagenase suspension, and then the digested, recovered adipose tissue is centrifuged to separate the floating adipocytes from free stromal-vascular fraction cells. A sample of the dissociated adipocytes is transferred from the recovered adipose tissue to a tube containing a vital stain to determine the percentage of viable adipocytes in the sample using an instrument capable of distinguishing live adipocytes from dead adipocytes based on the vital stain used. The results of the viability analysis will be reported to the collecting physician. Using this method the percentage of viable adipose tissue cells is typically greater than 70.0 percent.

The isolated material is distributed to the customer from which biological sample was obtained; and transporting the thawed container containing the cryoprotected adipose tissue in a transport system as directed to the request based on scheduled patient procedure.

Development of Direct Cryopreservation of Adipose Tissue

The freezing of adipose tissue was evaluated in four different cryopreservation conditions, two with the widely used dimethylsulfoxide (DMSO, in CryoStor CS-5 and CS-10, with 5% and 10% DMSO, respectively), and two with 10% glycerol, an older but still useful cryoprotectant, with and without 0.2M trehalose. Trehalose is a very stable disaccharide (sugar) that has been found to be useful in the cryopreservation of a wide variety of cell types, including adipose tissue. In fact, both glycerol and trehalose are produced endogenously as cryoprotectants by certain organisms. Pu et al. froze small samples of lipoaspirates in 3.3% DMSO+0.2M trehalose (2004), and subsequently in 0.2M trehalose alone (2005), and found that these worked well in terms of the post-thaw viability of the adipose tissue. We previously tested 10% DMSO, 7.5% DMSO+polyvinylpyrrolidone (PVP), 10% glycerol, and 10% glycerol with 10% FBS (2005), and found that cryopreservation with DMSO gave better results than glycerol (Moscatello et al., 2005). However, in those experiments we had not tested equilibration of the adipose tissue with the cryoprotectants prior to cryopreservation as we did in the current experiments. Time for equilibration is more likely to be an important variable with glycerol than with DMSO, as DMSO penetrates cells more rapidly than does glycerol (Jiang, 2008), but DMSO is toxic to cells at room temperature or body temperature, whereas glycerol is not toxic.

In the experiments performed, adipose tissue frozen in both glycerol and DMSO yielded similar post-thaw adipocyte viability (Table 1). This was established by rapidly thawing the cryopreserved adipose tissue (AT) in a 37° C. waterbath and washing with lactated Ringer's solution, followed by digestion with 1 mg/mL collagenase. The digested adipose tissue was washed once again, and the floating dissociated adipocytes were mixed with an equal volume of either acridine orange (AO) or propidium iodide (PI) stock solutions. The numbers shown in the table for both the AO and PI assays are the percent viable. In the case of the AO assay, this is the % AO positive (#AO+/#BR cells×100%), and in the case of the PI assay, is (#BR+−#PI+)/(#BR cells)×100%. In other words, in both assays, the bright field images are used to determine the total number of adipocytes counted. In the AO assay, the AO-positive cells are considered viable, and in the PI assay, the PI-positive cells are considered non-viable, but the purpose in both cases is to estimate the percent viability. The software does the calculations, which are specific to the dye being used.

TABLE 1
Viability after thawing of adipose tissue cryopreserved in various cryoprotectants.

| | Whole AT Recovery Percent Viability | | | | | | | |
|---|---|---|---|---|---|---|---|---|
| | Gly & Trehalose | | Gly in LR | | CS-5 | | CS-10 | |
| Series | AO | PI | AO | PI | AO | PI | AO | PI |
| 1 | 71.6% | 82.5% | 95.5% | 85.4% | 77.4% | 22.3% | 43.6% | 33.5% |
| | 98.6% | 85.8% | 94.0% | 87.5% | 82.6% | 76.3% | 71.2% | 70.0% |
| 2* | 73.2% | 93.4% | 95.4% | 32.0% | 83.6% | 74.5% | 74.3% | 86.1% |
| | 77.2% | 58.8% | 80.1% | 47.6% | 83.4% | 88.8% | 83.8% | 72.8% |

outlier - discrepant result

| | | | | | | | | |
|---|---|---|---|---|---|---|---|---|
| average | 80.2% | 80.1% | 91.3% | 63.1% | 81.76% | 65.48% | 68.2% | 65.6% |
| adj ave | 74.0% | 87.2% | 91.3% | 86.5% | 81.8% | 79.9% | 79.9% | 76.3% |

*Series 2 PI counts of both Gly in LR samples contained substantial extracellular matrix/stromal cells, potentially causing false positive F 1 counts.

Both assays work fairly well, but the PI assay is more sensitive to the amount of stroma (which can vary substantially) in the adipose preparation. This is because in preparations with a lot of stromal cells, some of which are non-viable, the nuclei of the stromal cells may overlap with adipocytes, and thus some viable adipocytes are counted as non-viable in such situations. Those of skill in the art will recognize while it is possible to digest and process adipose tissue sufficiently to completely separate the mature adipocytes from the stromal-vascular fraction cells, such a procedure is too harsh to retain the viability of the delicate adipocytes. The experiments, illustrated a larger volume of adipose tissue is required in the QC vials than might seem necessary from a theoretical standpoint. Although only about 100 μL of digested adipose tissue is needed to stain and load the deep-well slides, e.g. Cellometer slides, at least 2 mL of adipose tissue in the QC aliquot is required to ensure an equal volume of clean adipocytes suspension. Since the digestion to obtain viable adipocytes must be more gentle by virtue of the fragility of mature adipocytes than the digestion used to isolate SVF, any stromal material present is not well dissociated. This material must therefore be avoided or loading the slide is impossible, so we need sufficient volume to obtain at least 100 μL of essentially stroma-free adipocytes.

TABLE 2
Time course of cryopreserved adipose tissue viability after thawing.

| | Percent Viability | | | | | | Percent Viability | | | |
|---|---|---|---|---|---|---|---|---|---|---|
| | CS0T5 | | CS5T5 | | CS5T15 | | C10T5 | | CS10T15 | |
| | AO | PI | AO | PI | AO | PI | AO | PI | AO | PI |
| Day 0 | 59.4% | 64.5% | 96.6% | 68.6% | 50.1% | 63.2% | 42.9% | 67.2% | 45.9% | 40.7% |
| 24 | 55.8% | 68.4% | 62.2% | 89.2% | 93.7% | 84.1% | 76.8% | 48.6% | 18.0% | 14.2% |
| hrs | | | | | | | | | | |
| 48 | ND | ND | 87.5% | 97.1% | 69.3% | 88.1% | ND | ND | 91.2% | 61.8% |
| hrs | | | | | | | | | | |

All samples were from lipoaspirate waste aliquoted by RZG into OriGen bags on Nov. 17, 2011. After thawing, the Day 0 aliquot was removed, then an equal volume of Lactated Ringers was added to remaining adipose tissue and stored at 4° C. Very high connective tissue content, difficult to get ‘clean’ adipose tissue for count.
Legend: CS0 = no cryoprotectant; CS5 = CryoStor CS-5; CS10 = CryoStor CS-10; T5 = equilibration for 5 min; T15 = equilibtation for 15 min

TABLE 3

| | | | | | Percent Viability | | | |
|---|---|---|---|---|---|---|---|---|
| F02142012 | Percent Viability | | | | Gly + | | Gly + | |
| Freeze: | Glycerol—removed | | Glycerol—left in | | Tre—removed | | Tre—left in | |
| Assay: | AO | PI | AO | PI | AO | PI | AO | PI |
| Day 0 | 58.9% | 59.3% | 84.2% | 79.4% | 69.1% | 80.2% | 91.3% | 88.6% |
| Day 1 | 82.7% | 97.1% | 55.9% | 86.5% | 92.6% | 88.8% | 46.2% | 56.5% |
| NF* | | | | | | | | |
| Day 1 LR* | 94.3% | 95.9% | ND | ND | 73.9% | 90.7% | ND | ND |
| Day 2 | 77.6% | 98.0% | 86.1% | 87.2% | 86.5% | 92.6% | 63.0% | 86.1% |
| NF* | | | | | | | | |
| Day 2 LR* | 91.0% | 90.1% | 80.2% | 90.7% | 79.7% | 96.9% | 84.6% | 33.3% |

Samples were stored in Pall bags which hold a maximum of 25 mL. Samples were equilibrated in either 10% Glycerol in Lactated Ringer's (“Glycerol”) or 10% Glycerol in Lactated Ringer's + 7.6% Trehalose (“Gly + Tre”) for 15 minutes by rocking at 8 rocks/minute in the cold. Samples marked “Removed” were centrifuged for 3 minutes at 800 rpm, then the cyroprotectant was removed before adding the adipose tissue to the bags. Samples marked “Left in” were injected into Pall bags immediately after equilibration with the cryoprotectant. Final volumes were 12.5 mL adipose tissue and 12.5 mL cryoprotectant (“left in”) or 20 mL of Adipose tissue (“removed”). All samples were frozen on controlled-rate freezer Program ASC001 to −80 C. and stored in the vapor phase of the liquid nitrogen cryotank.
*NF—no fluid; LR—stored in Lactated Ringer's. ND = Insufficient volume of AT to test these conditions.
Note:
Almost all “cryoprotectant left in” samples had considerably higher amounts of stroma compared to “cryoprotectant removed”

Considering the results of the experiments shown in Tables 1, 2, and 3; 10% glycerol in Lactated Ringer's solution was chosen as the optimal cryoprotectant. The percentage of viable adipose tissue cells is greater than 70.0 percent as determined by acridine orange (AO) staining (average of all AO counts of thawed, glycerol-cryopreserved adipose samples in tables 1 and 3=80.1 percent, range 46.2 percent to 98.6 percent).

While the DMSO-based cryoprotectants worked comparatively well, any DMSO-based cryoprotectant would need to be washed out after recovery; in contrast this would not be necessary with glycerol based cryoprotectant of the present invention.

### Example 1

In a preferred example, the method is as follows:

Sterile containers containing Ham's F12 (HF12) medium with a broad-spectrum antibiotic (e.g., 50 μg/mL gentamicin sulfate) will be used for collection of adipose tissue. It should be clear to those skilled in the art that nutrient media such as Minimal Essential Medium, MCDB 201 or similar media, or isotonic solutions such as Hank's Balanced Salt Solution are contemplated as suitable alternatives to HF12. The containers will be weighed empty and after addition of the collection medium. The sterile container will be labeled with the client's name and other required information upon receipt of a collection request from the client's physician, and this will be shipped to the physician's office for receipt by the day prior to the scheduled liposuction. The shipment will be in a foam-insulated box of appropriate size, and will include patient and collection data forms and informed consent forms, as well as return shipping labels and a temperature monitor. The nature and size of the collection vessel will depend upon the volume of AT required:

Sterile bags (e.g., 150 mL Sartorius Flexboy Bags) containing 50 mL of Ham's F12 (HF12) medium with a broad-spectrum antibiotic (e.g., 50 μg/mL gentamicin sulfate) will be used for collection of small to moderate volumes of adipose tissue (up to 60 mL AT). The central luer cap on the bag will be replaced with a needleless access port (e.g., BD Q-Syte) for direct connection of a syringe to the bag for injection of AT by the physician.

In situation wherein, 300 mL to 2 L capacity of AT are collected, a Sterile Filtron lipoaspirate collection containers can be used. The Filtron collection vessel is designed to remove blood and tumescent fluid during the liposuction procedure. In this situation, a bag containing an appropriate volume (100 mL for a Filtron 300 up to 1 L for a 2 L Filtron) of sterile HF12 containing antibiotic will be supplied to add to the Filtron affer collection of the AT.

The lipoaspirate and associated paperwork will be returned to the processing laboratory by overnight shipment. Upon receipt, the sample will be inspected for container integrity and the sample and documents compared to ensure the correct sample was received.

The AT sample container will be weighed upon receipt and after removal of the shipping medium, so that the actual amount of AT received can be quantified.

The AT will be washed once with an equal volume of Lactated Ringer's solution.

An aliquot of this wash will be used for sterility quality control testing. After removal of the wash, the AT will be equilibrated with cryoprotectant and cryopreserved as follows:
a. A volume of 10% glycerol USP in Lactated Ringer's Injection USP., equal to the volume of AT to be preserved will be added to the container;
b. the container will be ‘sandwiched’ between two cold packs (previously chilled to 20-8° C.) and rocked at 8 rocks per minute for 15 minutes, alternatively, the rocker may be placed in a refrigerator or walk-in cold room to maintain the required temperature.
c. the container holding the equilibrated AT will be hung at about 4° C. for 10 minutes, and then the infranatant cryoprotectant fluid will be removed. Alternatively, the container may be centrifuged at 800 rpm for 3 minutes to separate the phases,
d. the infranatant comprising excess cryoprotectant fluid will then be removed, and portions thereof will be tested for the presence of microbial contaminants by inoculation into media suitable for the growth of both aerobic and anaerobic microorganisms;
e. the AT is then dispensed into cryogenic containers of appropriate size for the required volumes. Several versions of bags capable of holding from as little as 7 milliliters to as much as 100 milliliters of tissue are available which are certified as sterile, non-pyrogenic, and validated to retain integrity at liquid nitrogen temperatures. Most commonly, the inlet tubing on the cryogenic storage bags shall be no more than 10 inches in length, and preferably less than 5 inches in length. The bag shall have a needless luer-lock port for direct connection to a syringe for addition or removal of the AT. In a preferred embodiment, AT volumes of <100 mL will be dispensed into cryogenic storage bags capable of holding 20-25 mL at no more than 1 centimeter (cm) thickness. AT volumes ≧100 mL will be dispensed into the required number of cryogenic bags capable of holding 100 mL at no more than 1 cm thickness. The bags are then promptly placed into metal cassettes of sufficient size to contain each bag while ensuring that at no point is the vessel >1 cm in thickness. Both the bags and cassettes shall be labeled with a client-specific barcoded label. In another preferred embodiment, one or more quality-control samples of at least 2 mL will also be cryopreserved for each AT sample;
f. the AT will then be frozen in a controlled-rate freezer at about −1° C./minute. In a preferred embodiment, the freezing rate is −1° C./minute to −20 C, held for 0 to 10 minutes at −20° C., and then cooled at about −2° C./minute to −80° C. The cooling rate subsequent to the phase transition (from liquid to solid) is less critical than the initial cooling rate.
g. it will be apparent to those skilled in the art that many variations of such a freezing program are possible; and
h. the frozen AT samples will then be transferred to racks in the vapor phase of a liquid nitrogen cryogenic storage tank for long-term storage. The temperature must be maintained at or below −150 C. Most commonly, the temperature in the vapor phase shall optimally be between −180° C. and −190° C., and this will be continuously monitored.

Upon scheduling an autologous adipose tissue transfer procedure, the physician will submit a request to American CryoStem specifying the date and quantity of AT required. One or two days prior to the scheduled date, laboratory personnel will retrieve a quality control aliquot along with the requested AT sample(s) from cryogenic storage in the following manner.

The patient's AT bag will be rapidly thawed by removal from the metal cassette followed by immersion in a warm water bath with a temperature between 35 degree C. and 40.degree. C.; in a preferred embodiment, the water temperature will be about 37.degree. C. The quality control vial will also be thawed in the same way, but in both cases, the ports or cap will not be immersed. The containers will then be spiayed with 70% alcohol and wiped dry.

The bag(s) containing the AT will be placed between cold packs chilled as described herein in foam-insulated boxes, along with a temperature monitor. Also included will be a sterile, spike to Luer adaptor for the physician to use to remove the adipose tissue from the bag. These will then be shipped to the requesting physicians' office either overnight (if thawed two days before the scheduled procedure) or priority overnight (if thawed one day before the scheduled procedure).

On the day the adipose tissue is delivered to the physician, the thawed quality control aliquot will be processed washing and gentle digestion with collagenase enzyme, and tested for viability using vital dyes on a cell analysis instrument.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A cryoprotectant solution for cryopreserving biological tissue consisting essentially of:
a. a polyol; and
b. a crystalloid.

2. The cryoprotectant solution of claim 1, wherein the polyol is glycerol.

3. The cryoprotectant solution of claim 2, wherein the crystalloid is Lactated Ringers solution.

4. The cryoprotectant solution of claim 3, wherein the biological tissue is adipose tissue.

5. The cryoprotectant solution of claim 4, wherein the glycerol and Lactated Ringers are in a ratio of approximately 1 to 10 respectively.

6. The cryoprotectant solution of claim 5, wherein the solution establishes equilibration within 30 minutes after combination with adipose tissue.

7. A system for cryopreserving biological tissue comprising:
a. a cryoprotectant solution which does not cause leaching from a plastic based containers;
b. a plastic based container;

8. The system of claim 7, wherein the cryoprotectant solution comprises (i) a polyol and (ii) and (ii) a crystalloid.

9. The system of claim 8, wherein the polyol is glycerol.

10. The system of claim 9, wherein the crystalloid is Lactated Ringers solution.

11. The system of claim 10, wherein the plastic based container comprises at least one Luer tube port and at least one spike port.]

12. A method to obtain high percent viability of adipocytes after cryopreservation and thawing of adipose tissue comprising the steps of:
a. obtaining an adipose tissue specimen;
b. washing the adipose tissue with a wash solution comprising Lactated Ringer's solution;
c. adding a solution of glycerol in Lactated Ringer's to the washed adipose tissue to obtain glycerol cryoprotected adipose tissue in the original container and at least one second container;
d. cryopreserving the glycerol cryoprotected adipose tissue;
e. thawing the cryopreserved glycerol cryoprotected adipose tissue in a liquid bath at a temperature of approximately 37 degrees Celsius to form a recovered glycerol protected adipose tissue;
f. adding Lactated Ringers solution in an amount approximately equal to the volume of glycerol protected adipose tissue in the container to form a suspension;
g. separating the suspension to form (i) infranatants and (ii) adipose tissue;
h. removing the infranatants from the suspension
i. returning the recovered adipose tissue to the physician for use in a scheduled autologous adipose tissue transfer procedure;
j. recovering a quality-control aliquot of the same cryopreserved adipose tissue;
k. adding Lactated Ringers in an amount substantially larger than the volume of adipose tissue in the quality control aliquot;
l. centrifuging the re-suspended, recovered adipose tissue to form (i) infranatant wash and (ii) washed, recovered adipose tissue;
m. transferring an aliquot of the washed, recovered adipose tissue to a tube containing collagenase solution;
n. incubating the adipose tissue-collagenase suspension at approximately 37 degrees C. to partially dissociate the adipose tissue into adipocytes and stromal-vascular fraction cells;
o. neutralizing the collagenase by adding a growth medium to the adipose tissue-collagenase suspension: centrifuging the digested, recovered adipose tissue to separate the floating adipocytes from free stromal-vascular fraction cells;
p. transferring a sample of the dissociated adipocytes from the recovered adipose tissue to a tube containing a vital stain;
q. determining the percentage of viable adipocytes in the sample using an instrument capable of distinguishing live adipocytes from dead adipocytes based on the vital stain used; and
r. reporting the results of the viability analysis to the collecting physician.

13. The method of claim 12, wherein the percentage of viable adipose tissue cells is greater than 70.0 percent as determined by acridine orange (AO) staining.

14. The method of claim 13, wherein the defined rate of cooling is −1 degree Celsius/minute to at least −20 degrees Celsius, and cooling is continued at −1 to −2 degrees Celsius per minute to −80 degrees Celsius.

15. A method cyropreserve adipose tissue comprising the steps of:
a. obtaining an adipose tissue specimen;
b. washing the adipose tissue with a wash solution comprising a crystalloid solution;
c. removing the wash;
d. adding a cryoprotectant solution comprising a polyol and a crystalloid equal to the volume of adipose tissue to be preserved;
e. separating and removing infranatant solution;
f. testing the infranatant solution for microbial contamination;
g. placing the cryoprotected adipose tissue into multiple containers;
h. cryopreserving the cryoprotected adipose tissue at a defined rate; and
i. storing the cryopreserved adipose tissue at a temperature below −150 degrees Celsius.

16. The method of claim 15, wherein the defined rate of cyropreserving is −1 degree Celsius/minute to at least −20 degrees Celsius, and cooling is continued at −1 to −2 degrees Celsius per minute to −80 degrees Celsius. [EXPLAIN] The cooling rate subsequent to the phase transition (from liquid to solid) is less critical than the initial cooling rate.

17. The method of claim 16, wherein the polyol is glycerol.

18. The method of claim 17, wherein the crystalloid is Lactated Ringers solution.

19. A business method to obtain high percent viability of adipocytes after cryopreservation and thawing of adipose tissue for transportation and use in patient procedures comprising the steps of:
a. collecting a premium for defined services for collection, transportation, cryogenic storage and distribution of a biological sample material;
b. coordinating the collection of a biological sample of a customer comprising (i) paying a predetermined fee in support of physician services performed for collection of the biological sample and (ii) supplying a collection system comprising a plurality of components for collection and transportation of the biological sample;
c. obtaining the biological sample from the client;
d. transporting the biological sample in the collection system to a processing facility;
e. introducing information from the collection system components to a processing module of a database;
f. cryoprotecting the biological sample in a solution comprising (i) glycerol and (ii) Lactated Ringers for cryopreservation;
g. testing for quality control of the isolated material for cryopreservation;
h. cryopreserving the isolated material;
i. thawing the cryopreserved biological sample;
j. distributing the isolated material is to the customer from which biological sample was obtained; and
k. transporting the thawed container containing the cryoprotected adipose tissue in a transport system as directed to the request based on scheduled patient procedure.

20. The business method of claim 18, wherein the collection system comprises:
a. identification material for the obtained biological sample;
b. coded labels for use with an encoded program;
c. client sample bags;
d. transportation box; and
e. transportation labeling.

21. The business method of claim 2, wherein the collection system components are introduced to a processing module of a database via a log-in port, by scanning a barcode on the client sample bag in the completed recording information.

22. The business method of claim 3, wherein the obtained biological sample is an adipose tissue sample.
